(19) United States
(12) Patent Application Publication
(10) Pub. No.: US 2026/0347950 A1
(43) Pub. Date: Nov. 5, 2026

(54) METHODS OF SELECTING PREBIOTICS OPTIMIZED FOR AN INDIVIDUAL WITH INFLAMMATORY BOWEL DISEASE

(71) Applicant: UNIVERSITY OF OTTAWA, Ottawa (CA)

(72) Inventors: Alain Christophe STINTZI, Ottawa (CA); Joseph Michel Daniel FIGEYS, Ottawa (CA); David R. MACK, Ottawa (CA); Peter DOBRANOWSKI, Ottawa (CA)

(21) Appl. No.: 19/488,393

(22) PCT Filed: May 28, 2024
(86) PCT No.: PCT/CA2024/050708

§ 371 Date: Nov. 26, 2025

Related U.S. Application Data
(60) Provisional application No. 63/469,479, filed on May 29, 2023.

(51) Int. Cl. C12Q 1/689; C12Q 1/04

(52) U.S. Cl. CPC C12Q 1/689; C12Q 1/04; C12Q 2600/136

(57) ABSTRACT

Methods of selecting prebiotics, optionally resistant starch, optimized for an individual with inflammatory bowel disease and methods of treating individuals with the selected prebiotics are provided.

### FIELD OF THE INVENTION

The present application relates generally to prebiotic compositions including resistant starches and, more particularly, to methods of selecting prebiotics optimized for an individual with inflammatory bowel disease.

### BACKGROUND OF THE INVENTION

Inflammatory bowel disease (IBD) is a significant health and economic burden. Canada has one of the highest rates of IBD in the world, with >10,200 newly diagnosed patients each year added to the estimated 1 in 135 Canadians with IBD. IBD's projected cost to the Canadian economy is $2.8 billion/year (including direct medical costs of $1.2 billion/year). IBD is an incurable, life-long disease characterized by remitting and relapsing mucosal inflammation and encompasses two main conditions: ulcerative colitis (UC) and Crohn's disease (CD). 15-20% of cases are diagnosed in childhood (<18 years), and the incidence of pediatric IBD is growing in developing countries. Due to the early onset of disease, and the propensity for pediatric IBD patients to present with severe disease, children with IBD have an elevated risk of long-term comorbidities such as colorectal cancer.

Dysbiosis in the gut microbiome can contribute to disease and is commonly observed in patients with IBD. The products of gut microbiome fermentation include short chain fatty acids such as propionate, acetate and butyrate, as well as other metabolites, that can impact intestinal homeostasis and immune function. For example, microbial-derived butyrate promotes IL-10 independent epithelial barrier function and healthy gut immunity. The gut microbiome of patients with active IBD have reduced butyrate biosynthetic-capacity. Other microbial metabolites including secondary bile acids, aromatic amino acid catabolites and sphingolipids may also impact gut health.

Resistant starch (RS) are granular alpha-glucans capable of withstanding hydrolysis by human amylases. As a common component of foods and supplements, RS possess diverse morphological, physicochemical, and fermentative properties, principally conferred by plant origin and/or hydrothermal/chemical processing [reviewed in ¹]. Upon transit to the colon, microbial RS fermentation is considered a collaborative effort initiated by RS-active microbes (the primary degraders) liberating glycans to be transformed by cross-feeders, contributing to a cascade of ecosystem remodelling².

Fermentation of prebiotics, RS included, has been studied with an emphasis on short-chain fatty acid (SCFA) production, foremost butyrate³⁻¹⁴. Butyrate plays important roles in maintaining host gut and systemic homeostasis, with potential therapeutic ramifications in conditions such as inflammatory bowel disease (IBD)^{1,15}.

A systematic review and meta-analysis of the preclinical and clinical effects of RS on IBD suggests that RS is associated with reduced histological damage in animal studies, and improvements in IBD patient clinical remission²⁵. However, it is important to consider these findings in light of the potential bias present in the included studies. In particular, human trials showed significant variability in treatment effect. These varied outcomes are likely due to each RS having a different interaction with each individual's microbiota composition and/or functionality.

Accordingly, there is an urgent need for new treatments for IBD including a need for personalized dietary intervention that includes personalized RS based intervention.

### SUMMARY OF THE INVENTION

An object of the present invention is to provide methods of selecting prebiotics, such as resistant starch, personalized or optimized for an individual with inflammatory bowel disease.

In accordance with an aspect of the invention, there is provided a method of selecting prebiotic nutrition therapy, optionally a resistant starch therapy for a subject having inflammatory bowel disease, the method comprising providing a plurality of gut microbiome samples, optionally stool samples, from one or more subjects; culturing the plurality of gut microbiome samples in conditions that replicate the lumen environment of a colon or small intestine; supplementing each gut microbiome sample in the plurality of stool microbiome samples with a prebiotic for a period of time; analyzing the gut microbiome's response to the prebiotic; and selecting the optimal prebiotic nutrition based on the analyzed changes.

In some embodiments, analyzing the gut microbiome responses comprise metabolomic, metaproteomic, metatranscriptomic and/or metagenomic analysis comparing pre and post treatment.

In some embodiments, the prebiotic nutrition is selected to increase butyrate producing microbes or other beneficial microbes including Faecalibacterium, Ruminococcus, Eubacterium, Lachnospiraceae, Roseburia, Clostridium and Blautia; and combinations thereof.

In some embodiments, the prebiotic nutrition is selected to decrease abundance of pathobionts including Veillonella, Atopobium, Fusobacterium, Leptotrichia, Prevotella, Haemophilus and Streptococcus. Optionally, in some embodiments, pathobionts further includes one or more of Escherichia, Salmonella, Shigella and Klebsiella.

In accordance with another aspect of the invention, there is provided a method of treating inflammatory bowel disease in a subject comprising of administering a therapeutically effective amount of resistant starch, wherein the resistant starch is selected by a method comprising: culturing a plurality of gut microbiome samples, optionally stool samples, from the subject in conditions that replicate lumen environment of a colon or small intestine; supplementing each gut microbiome sample in the plurality of gut microbiome samples with a prebiotic for a period of time; analyzing the gut microbiome's response to the prebiotic; and selecting the optimal prebiotic nutrition based on the analyzed changes.

### BRIEF DESCRIPTION OF THE DRAWINGS

These and other features of the invention will become more apparent in the following detailed description in which reference is made to the appended drawings.

FIG. 1 illustrates that resistant starch fermentation is starch- and individual-specific. Ex vivo cultured microbiome supernatant changes in pH relative to negative control (PBS) for 9 resistant starches (RS) and 15 individual microbiomes, plotted across RS (A) and per individual (B). Changes in acetate, propionate, and butyrate plotted across RS (C) and per individual (D). Changes in observed microbial (richness (Observed) and diversity (Shannon index) plotted across RS (E) and per individual (F). Linear mixed effect model; *Bonferroni-adjusted p-value<0.05.

FIG. 2 illustrates that resistant starch fermentation remodels microbiome compositions. (A) Heatmaps of agglomerated bacterial taxa log-fold changes (LFC) relative to negative control (PBS) for 9 resistant starches (RS) and 15 individual microbiomes, plotted across RS. Features with the top 20 absolute log fold change are shown. (MaAsLin2; *Benjamini-Hochberg-adjusted p-value<0.05). (B) Principal coordinate analysis of Bray-Curtis dissimilarities in microbial compositions, grouped by individual. Variance explained by individual R²=68.85% (p=0.001) and individual R²=0.61% (p=0.025) using). PERMANOVA with 999 permutations. (C) Heatmaps of bacterial log-fold changes plotted per individual (MaAsLin2; *Benjamini-Hochberg-adjusted p-value<0.05), depicting taxa with the lowest Gini entropy (highest agreement across individuals) (C) and highest Gini entropy (lowest agreement across individuals) (D).

FIG. 3 illustrates that starch fermentation remodels microbiome functions. (A) Heatmaps of proteins collapsed to Cluster of Orthologous Group (COG) log-fold changes relative to negative control (PBS) for 9 resistant starches (RS) and 15 individual microbiomes, plotted across RS. Features with the top 20 absolute log fold change are shown (MaAsLin2; *Benjamini-Hochberg-adjusted p-value<0.05). (B) Principal component analysis of protein intensities, grouped by individual. Variance explained by individual R²=92.27% (p=0.001) and individual R²=0.06% (p=0.760) using). PERMANOVA with 999 permutations. (C) Heatmaps of COG log-fold changes plotted per individual, depicting COGs with the lowest Gini entropy (highest agreement across individuals) (C) and highest Gini entropy (lowest agreement across individuals) (D). MaAsLin2; *Benjamini-Hochberg-adjusted p-value<0.05.

FIG. 4 illustrates that resistant starch fermentation remodels microbiome metabolites (A) Heatmaps of metabolite log-fold changes relative to negative control (PBS) for 9 resistant starches (RS) and 15 individual microbiomes, plotted across RS. Features with the top 20 absolute log fold change are shown (MaAsLin2; *Benjamini-Hochberg-adjusted p-value<0.05). (B) Principal component analysis of metabolite abundances, grouped by individual and colored by RS. Variance explained by individual R²=82.00% (p=0.001) and individual treatment R²=9.53% (p=0.001) using). PERMANOVA with 999 permutations. (C) Heatmaps of metabolite log-fold changes plotted per individual, depicting metabolites with the lowest Gini entropy (highest agreement across individuals) (C) and highest Gini entropy (lowest agreement across individuals) (D) (MaAsLin2Maaslin2; *Benjamini-Hochberg-adjusted p-value<0.05).

FIG. 5 illustrates that cross-omic feature interactions are resistant starch- and individual-specific. Networks depicting significant correlations between bacteria, proteins, and metabolites (Spearman correlation; Benjamini-Hochberg-adjusted p-value<0.05). Features were filtered to those identified as significantly altered relative to negative control (PBS) using MaAsLin2. Samples were filtered to negative control (PBS) and each RS independently for Spearman coefficient calculation for each individual.

FIG. 6 illustrates that commercial resistant starch preparations have differing percentages of RS. Resistant starch and control starch (Amioca) product RS %. Median values are labeled.

FIG. 7 illustrates an example selection of the optimal RS for 9 IBD patients. The RS therapeutic adjuvant selection algorithm consists of 3 steps. First step: Participant microbiotas are cultured in optimized medium containing RS in a 96-well deep-well plate (one specific RS per well) at 37 °C under strict anaerobic conditions for 18 hr followed by metagenomics analyses. Second step: Butyrogenic bacteria relative abundances are calculated for each RS treatment and these data are converted to Z-scores. Step 3: The RS treated microbiomes are compared to those from control participants. RS with a Z-score>1 (from step 2) are evaluated to identify the RS that shifts the microbiota closest to a control cohort and this RS is selected as the optimal RS to use as a therapeutic adjuvant. The selected RS is indicated at the top of the graph representing the Z-score analysis for each participant.

FIG. 8 illustrates decreased expression of the butyrate COG pathway correlates with increasingly aberrant microbiota composition. Plotting the median butyrate COG abundance against the microbiota's median distance from controls reveals that microbiotas become increasingly aberrant as butyrate pathway expression decreases. Spearman rho and p-value are given inside the plot. The line represents a linear regression.

FIG. 9 illustrates that butyrogenic bacteria are negatively correlated with IBD pathobionts. The relative abundances of butyrate-producing bacteria are negatively correlated (Spearman correlation) with the relative abundance of pathobionts. Spearman rho and p-value are given inside the plot. The blue line represents a linear regression of the data.

FIG. 10 illustrates RS selection frequency. The RS selection algorithm takes into account two key factors: butyrogen relative abundances and dissimilarity to a non-IBD reference cohort. The relative abundances of butyrate-producing taxa from the RapidAIM tests are quantified for each patient (x axis), and standardized into Z-scores, generating butyrogen enrichment values for each RS (y axis). Second, within the RS with butyrogen Z-scores>1, the RS that results in a microbiome composition which best correlates with non-IBD microbiomes chosen as likely to elicit the best response in the patient and prescribed to the patient. The dot represents the RS source and those with black outlines indicate the RS starch prescribed to each patient as summarized in the frequency graph.

FIG. 11 illustrates the RS-RapidAIM process.

FIG. 12 illustrates characteristics of the cohort's microbiome at RS optimization and effects of RS on fecal bacterial diversity for a subset of the participants.

FIG. 13 illustrates effects of RS on fecal bacterial diversity.

FIG. 14 illustrates butyrate and H₂S metabolic pathways are altered in IBD and effects of RS on bacterial species (only four species are shown).

### DETAILED DESCRIPTION OF THE INVENTION

The present invention provides methods of selecting prebiotics, including resistant starches (RS), optimized to improve the gut microbiome and/or restore a healthy gut microbiome in an individual with inflammatory bowel disease by pre-screening a panel of RS for one or more RS that have a therapeutically positive impact on the ex vivo gut microbiome of the individual. In some embodiments, the method further comprises treating the individual with the selected RS for a period of time and rescreening for optimized prebiotics after this period of time. In some embodiments, the suitable period of time may be for example about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, 3 months, 4 months, 5 months, 6 months, 7 months, 8 months, 9 months, 10 months, 11 months, 12 months or longer.

In some embodiments, re-screening occurs following a period of about 3 months.

Accordingly, in some embodiments, the method is repeated after a treatment period to further optimize the RS in view of changes in the patient's gut microbiome in response to the current optimized RS. Optionally, the method is repeated at regular intervals and adjustments to the treatment regime including changes to the RS made as necessary.

Individuals with inflammatory bowel disease (IBD) may have Crohn's Disease or Ulcerative Colitis or undefined inflammatory bowel diseases. The individual with IBD may be a pediatric patient or an adult patient, optionally the individual is undergoing or has undergone medical, surgical, and/or dietary therapy for IBD. RS treatment is used as an adjuvant therapy. In some embodiments, the RS treatment is for use with fecal transplant, or with probiotic or prebiotic therapies.

In some embodiments, the individual with IBD is in a period of remission or mild disease. Optionally, RS treatment is implemented during remission induction or immediately after remission is induced.

A therapeutically positive impact includes one or more of restoration of gut microbiome homeostasis, increased abundance of butyrate producers and/or other short chain fatty acid producers, decrease in prevalence of pathobionts, decreased intestinal mucosal inflammation, decreased fecal calprotectin, restoration of mitochondrial function, improvement in clinical disease activity, and/or improvement of endoscopic disease activity.

In some embodiments, a therapeutically positive impact is assessed based on a comparison of gut microbiome composition, metabolites, microbial (including bacterial) protein or RNA expression with those of a healthy gut microbiome or an individual with inflammatory bowel disease in a period of remission.

In some embodiments, a therapeutically positive impact is assessed based on a comparison of host protein or RNA expression with those of a healthy individual or an individual with inflammatory bowel disease in a period of remission.

In some embodiments, a therapeutically positive impact is assessed based on a comparison of metabolites with those metabolites produced by a healthy gut microbiome. Optionally, metabolites assessed include short chain fatty acids including acetate, propionate and butyrate, secondary bile acids, aromatic amino acid catabolites, sphingolipids, and amino acids. In such embodiments a therapeutically positive impact is an impact that results in a metabolites profile more similar to or substantially similar to a metabolite profile of a healthy gut microbiome.

In some embodiments, a therapeutically positive impact is assessed based on changes in intestinal mucosal inflammation by measuring fecal calprotectin through stool samples, change in endoscopic disease activity measured during colonoscopies including using the SES-CD for Crohn's Disease and the Mayo Endoscopic Sub Score and UCEIS for Ulcerative Colitis, change in clinical disease activity as measured by the PCDAI/wPCDAI for Crohn's Disease, the PUCAI and Partial Mayo Score for Ulcerative Colitis, the Harvey Bradshaw and CDAI scores, and the PGA for both Crohn's Disease and Ulcerative Colitis, and changes in histological scoring of acute and chronic inflammation assessed using Naini and Cortina score for Crohn's disease and the Robarts Histopathological Index (RHI) for Ulcerative Colitis. In such embodiments a therapeutically positive impact is an impact that results in improved disease activity scores and/or inflammation by comparing pre-treatment measurements to those taken during and after treatment.

Resistant starch (RS) refers to starch that remains undigested and reaches the large intestine. The resistance exhibited by RS is determined by various physicochemical characteristics. These include its physical encasement within non-digestible substances (RS type 1), maintenance of its native supramolecular structure and morphology (RS type 2), retrogradation achieved through hydrothermal-cycling (RS type 3), chemical modifications predominantly involving ester cross-linking (RS type 4), and the formation of amylose-lipid complexes during cooking (RS type 5).

Examples of RS include but are not limited to: RS2 including but not limited to those found in green bananas, raw potatoes and high amylose corn starch, RS3 including but not limited to those found in cooked and cooled rice, potatoes and wheat, and RS4 including but not limited to starches that have been chemically modified to resist digestion. Isolated RS include but are not limited to RS2 resistant starch from high amylose corn or high amylose wheat, RS3 resistant starch from cassava and RS4 resistant starch from wheat and potato.

RS is provided in a therapeutically effective amount as would be appreciated by one of skill in the art. The “therapeutically effective amount” may be taken on a regular schedule or regimen, for example, once per day or every other day. “Therapeutically effective amount” includes single doses or multiple doses.

RS may be provided in a variety of forms including but not limited to capsules, tablets, chewables or powders. In some embodiments, RS is provided as a powder that can be added to food.

In some embodiments, the total daily therapeutically effective amount is about 5 to about 30 grams daily, about 10 to about 25 grams and about 15 to about 20 grams daily. In some embodiments, the dose of RS is increased gradually over a period of time until a final therapeutically effective amount is reached.

In some embodiments, the total daily therapeutically effective amount is determined based on the patient's body surface area [BSA]. In some embodiments, the dose of RS/m² is increased gradually over a period of time until a final therapeutically effective amount is reached. Optionally, in some embodiments, total daily dose is evenly divided into multiple doses.

In embodiments, where the RS is formulated as a chewable or powder, capsule and/tablet, flavoring or other additives may be provided to increase palatability.

In some embodiments, RS is provided in combination with fiber optionally including soluble and insoluble fiber.

In some embodiments, gut microbiomes are obtained from fecal samples. In other embodiments, gut microbiome samples are obtained using endoscopic approaches and thus region-specific samples. In such embodiments, gut microbiome samples are optionally obtained from one or more inflamed sites and one or more non-inflamed sites.

The gut microbiome includes bacteria, archaea, fungi, yeast, protozoa, phages and viruses with the microbial composition being dependent on location within the gastrointestinal tract. Bacterial species are predominant in the gut microbiome of the colon. Accordingly, in some embodiments, analysis of gut microbiome composition and diversity is limited to bacterial composition and diversity.

Methods of assessing gut microbiome composition are known in the art and include various metagenomic approaches including next generation sequencing based methods. These include whole and/or partial sequencing of marker genes including eukaryotic 18S rRNA and prokaryotic marker genes such as the bacterial 16S/23S rRNA genes (including the 16S V4 variable region and/or other variable regions), and/or other conserved genes (rpoB, cpn, gyrB); metagenomic sequencing and RNA sequencing, reverse-transcriptase polymerase chain reaction (RT-PCR)-based methods; quantitative PCR (qPCR) based methods and single nucleotide polymorphism based methods such as GenoTyper for Prokaryotes (GT-Pro).

Other methods of quantitively determining gut microbiome composition or levels of specific microbes therein are known in the art and include reverse-transcriptase polymerase chain reaction (RT-PCR)-based methods; quantitative PCR (qPCR) based methods; by microarray analysis using probes targeting specific taxa and/or phylogenetically related taxa, by quantitative fluorescent in situ hybridization (FISH) with probes recognizing microbe specific sequences, or by antibody or cell-binding based methods.

Microbes assessed included but are not limited to Achromobacter spp, Acidaminococcus fermentans, Acinetobacter calcoaceticus, Actinomyces spp, Actinomyces viscosus, Actinomyces naeslundii, Aeromonas spp, Aggregatibacter actinomycetemcomitans, Akkermansia spp, Akkermansia muciniphila, Anaerobiospirillum spp, Alcaligenes faecalis, Arachnia propionica, Atopobium parvulum, Atopobium spp., Bacillus spp, Bacteroides spp, Bacteroides gingivalis, Bacteroides fragilis, Bacteroides intermedius, Bacteroides melaninogenicus, Bacteroides pneumosintes, Bacterionema matruchotii, Bilophila wadsworthia, Corynebacterium matruchotii, Bifidobacterium spp, Buchnera aphidicola, Butyriviberio fibrosolvens, Campylobacter spp, Campylobacter coli, Campylobacter jejuni, Campylobacter sputorum, Campylobacter upsaliensis, Candida albicans, Capnocytophaga spp, Clostridium spp, Citrobacter freundii, Clostridium difficile, Clostridium sordellii, Clostridium clostridioforme, Corynebacterium spp, Cutibacterium acnes, Eikenella corrodens, Enterobacter cloacae, Enterococcus spp, Enterococcus faecalis, Enterococcus faecium, Escherichia coli, Escherichia spp., Eubacterium spp, Faecalibacterium spp, Faecalibacterium prausnitzii, Flavobacterium spp, Fusobacterium spp, Fusobacterium nucleatum, Gordonia spp, Haemophilus spp, Haemophilus parainfluenzae, Haemophilus paraphrophilus, Klebsiella pneumoniae, Klebsiella spp, Lactobacillus spp, Leptotrichia buccalis, Methanobrevibacter smithii, Morganella morganii, Mycobacteria spp, Mycoplasma spp, Micrococcus spp, Mycoplasma spp, Mycobacterium chelonae, Neisseria spp, Neisseria sicca, Peptococcus spp, Peptostreptococcus spp, Plesiomonas shigelloides, Porphyromonas gingivalis, Propionibacterium spp, Providencia spp, Pseudomonas aeruginosa, Roseburia spp, Rothia dentocariosa, Ruminococcus spp, Ruminococcus bromii, Ruminococcus gnavus, Sarcina spp, Salmonella spp., Salmonella enterica, Shigella spp., Staphylococcus aureus, Staphylococcus epidermidis, Streptococcus anginosus, Streptococcus mutans, Streptococcus oralis, Streptococcus pneumoniae, Streptococcus sobrinus, Streptococcus viridans, Torulopsis glabrata, Treponema denticola, Treponema refringens, Veillonella spp, Veillonella parvula, Veillonella dispar, Vibrio spp, Vibrio sputorum, Wolinella succinogenes, and Yersinia enterocolitica

In some embodiments, bacteria assessed include Bacteroides fragilis, Bacteroides melaninogenicus, Bacteroides oralis, Enterococcus faecalis, Escherichia coli, Enterobacter sp. Klebsiella sp., Bifidobacterium bifidum, Staphylococcus aureus, Lactobacillus spp., Clostridium perfringens, Proteus mirabilis, Clostridium tetani, Clostridium septicum, Pseudomonas aeruginosa, Salmonella enterica, Faecalibacterium prausnitzii, Peptostreptococcus spp., and Peptococcus spp.

In some embodiments, bacteria genera assessed include Lachnospiraceae, Blautia, Roseburia, Eubacterium, Ruminococcus, Clostridium, and Faecalibacterium.

In some embodiments, bacteria genera assessed include Veillonella, Atopobium, Fusobacterium, Leptotrichia, Prevotella, and Streptococcus.

In some embodiments, bacteria genera assessed include Veillonella, Atopobium, Fusobacterium, Leptotrichia, Prevotella, Streptococcus, Klebsiella, Escherichia, and Ruminococcus gnavus.

In some embodiments, the method comprises comparing the impact of each RS on microbiome diversity, community structure and/or taxa relative abundances. Microbiome composition may be compared to no treated IBD patients, non-IBD controls and/or healthy controls. Methods of comparing microbiome composition and structure are known in the art and include Bray-Curtis dissimilarity, alpha diversity (commonly used alpha diversity metrics include the Shannon index, Simpson index, Chao1 index, and observed species), beta diversity (unweighted UniFrac distance, Jaccard index, weighted UniFrac distance, Bray-Curtis dissimilarity), Permutational Multivariate Analysis of Variance (PERMANOVA), Principal Component Analysis (PCA), Principal Co-ordinate analysis (PCoA), Non-Metric Multidimensional Scaling (NMDS), and differential abundance analysis (various statistical approaches, such as DESeq2, edgeR, metagenomeSeq, MaAsLin2, ANCOM, ALDEX2, or LEfSe).

In some embodiments, the method comprises comparing the impact of different RS on microbialmetabolites. Methods of assessing metabolites are known in the art and include untargeted, semi-targeted and targeted metabolomic approaches. In some embodiments, the method comprises using a quantitative targeted metabolomics approach.

In some embodiments, levels of metabolites are determined by mass spectrometry, by nuclear magnetic resonance spectroscopy, by enzyme-linked immunosorbent assay (ELISA), by cell based assays, by colorimetry and/or by spectrophotometry. Optionally, metabolomic approaches may utilize standards.

In some embodiments, the method comprises use of High Performance Liquid Chromatography coupled with High-Resolution Mass Spectrometry (HPLC-HRMS).

In some embodiments, the method may further include identifying one or more metabolites in the extracted sample using a quantitative untargeted metabolomics assessment.

In some embodiments, the metabolites assessed include short chain fatty acids including acetate, propionate and butyrate, secondary bile acids, aromatic amino acid catabolites, sphingolipids, and/or amino acids.

In some embodiments, the metabolites assessed include metabolites of protein fermentation.

In some embodiments, the metabolites assessed included uridine, N-acetyl-cysteine, tyrosine, guanosine, gulonic acid gamma-lactone, hypoxanthine, adenine, uracil, cysteine, alpha-glucose, inosine, aspartate, 3-sulfino-alanine, guanine, pyridoxamine, xanthosine, thymidine, methionine, thymine, malate, tryptophan, cytidine, deoxyuridine, N-acetyl-glutamic acid, succinate, and 3-alpha-hydroxy-5-beta-cholanate.

In some embodiments, the metabolites assessed include N-acetyl-cysteine, malate, ribose, gulonic acid gamma-lactone, riboflavin, quinate, N-acetyl-aspartic acid, deoxyuridine, acetoacetate, uridine, urate, N-acetyl-methionine, pipecolic acid, glycerol 2-phosphate, cholate, alpha-ketoglutaric acid, 4-methylcatechol, 3-hydroxybutanoic acid, 3-dehydroshikimate, and 2′-deoxyguanosine.

In some embodiments, the metabolites assessed included butyrate, acetate or propionate.

In some embodiments, the metabolites assessed include [2-aminoethyl]phosphonate, glyceraldehyde, 3-sulfino-alanine, allantoin, selenomethionine, glucosamine, o-succinyl-homoserine, adenosine 5′-diphosphate, 5-oxo-proline, glucuronic acid, galactitol, paraxanthine, 4-pyridoxate, kynurenine, 1-methyladenosine, trans-4-hydroxyproline, 4-acetamidobutanoate, urocanate, dopamine, deoxyuridine, phosphonoacetate, 1-aminocyclopropane-1-carboxylate, 5-hydroxymethyluracil, 3-hydroxy-3-methylglutarate, arginine, cystathionine, xanthosine, thyrotropin releasing hormone, 4-hydroxy-proline, n-methyl-aspartic acid, n-methyl-glutamate, 6-deoxy-galactose, histidine, homocystine, thymidine-5′-diphospho-alpha-d-glucose, beta-nicotinamide adenine dinucleotide phosphate, n-alpha-acetyl-asparagine, glucose 6-phosphate, adenosine 5′-monophosphate, 2′-deoxyguanosine, 5′-monophosphate, nicotinamide hypoxanthine dinucleotide, cysteic acid, n-acetylglycine, alpha-hydroxyisobutyric acid, gluconate, 6-phosphogluconic acid, n-acetyl-aspartic acid, adenosine 2′-3′-cyclic monophosphate, uridine 5′-diphosphoglucose, mevalolactone, guanosine 5′-diphosphoglucose, phospho[enol]pyruvic acid, glyceric acid, dihydroxyacetone phosphate, pyridoxal 5′-phosphate, mandelic acid, cytidine 2′-3′-cyclic mono-phosphate, adenosine 3′-5′-cyclic monophosphate, thymidine 5′-monophosphate, adenosine 3′-5′-diphosphate, adenosine-5′-diphosphoglucose, 2-aminoethyl dihydrogen phosphate, o-acetyl-serine, fructose 6-phosphate, acetyl-lysine, 3-nitro-tyrosine, N-acetylneuraminate, Nicotinamide adenine dinucleotide, hypotaurine, threonine, glutamine, purine, urate, xanthosine 5′-monophosphate, lauroylcarnitine, 5-aminolevulinic acid, pyridoxamine, 2′-deoxyuridine 5′-mono-phosphate, guanosine 3′-5′-cyclic monophosphate, s-[5′-adenosyl]-homocysteine, o-phospho-serine, ornithine, n-acetyl-methionine, 5-aminoimidazole-4-carboxamide-1-beta-ribofuranosyl 5′-monophosphate, guanosine 5′-diphosphate, uridine 5′-diphosphoglucuronic acid, uridine 5′-diphospho-N-acetylglucosamine, indoxyl sulfate, cytidine 5′-diphosphate, guanosine 5′-monophosphate, thiamine monophosphate, s-hexyl-glutathione, glyoxylic acid, 4-guanidinobutanoate, 2-4-dihydroxypyrimidine-5-carboxylic acid, N-acetyl-alanine, mannosamine, 5′-deoxyadenosine, glutathione, guanosine 5′-diphospho-mannose, riboflavin, erythritol, glucosaminate, methyl beta-galactoside, CMP, inosine 5′-monophosphate, 2′-deoxyadenosine, 2-amino-2-methylpropanoate, N-acetyl-cysteine, 2-4-dihydroxypteridine, 6-hydroxynicotinate, N-acetyl-glutamic acid, N-acetyl-galactosamine, pantothenic acid, adenosine, thiamine pyrophosphate, raffinose, N-formylglycine, 3-ureidopropionate, histidinol, rhamnose, alpha-glucose, 3-methoxy-tyrosine, lactose, 3-hydroxybutanoic acid, methionine sulfoximine, 4-imidazoleacetic acid, cis-4-hydroxy-proline, galacturonic acid, allose, 5-hydroxy-tryptophan, maleamate, formyl-methionyl peptide, anserine, melibiose, adenosine 5′-diphosphoribose, creatine phosphate, citramalate, N-amidino-aspartate, 5-hydroxylysine, inosine, taurine, cysteine, serine, glucono-1-5-lactone, citrulline, isoleucine, nicotinate, cytidine, ophthalmic acid, 2-hydroxybutyric acid, sorbitol, trigonelline, 3-4-dihydroxyphenyl glycol, epinephrine, 3-hydroxykynurenine, stachyose, sn-glycerol 3-phosphate, ribose 5-phosphate, N-acetyl-serine, tagatose, galactosamine, 4-hydroxy-phenylglycine, 2′-deoxycytidine 5′-diphosphate, glycerol 2-phosphate, xylose, ribitol, sorbose, glucosamine 6-sulfate, alpha-glucose 1-phosphate, anthranilate, xanthurenic acid, cortisol, mono-ethyl malonate, 3-methyglutaric acid, psicose, hydroxybenzaldehyde, 4-hydroxybenzoate, 4-hydroxy-2-quinolinecarboxylic acid, maleic acid, 2-methylpropanoate, lyxose, N-acetyl-phenylalanine, ribose, glutarate, mannose 6-phosphate, 6-carboxyhexanoate, benzoate, 4-hydroxyphenylacetate, caffeate, 3-5-diiodo-tyrosine, ferulate, palatinose, glycocholate, propenoate, thiopurine s-methylether, 2-3-dihydroxybenzoate, 10-hydroxydecanoate, 2-hydroxypyridine, catechol, 3-4-dihydroxybenzoate, 3-amino-5-hydroxybenzoic acid, guaiacol, 2-hydroxyphenylacetic acid, indole-3-acetamide, n6-[delta2-isopentenyl]-adenine, serotonin creatinine sulfate complex, cortisol 21-acetate, 2-oxobutanoate, methylmalonate, 3-hydroxyanthranilate, lipoamide, 3-4-dihydroxyphenylacetate, 2-quinolinecarboxylic acid, 3-5-diiodo-thyronine, fumarate, ethylmalonic acid, pterin, 3-[2-hydroxyphenyl]propanoate, hippurate, glycolate, malonate, malate, dihydroorotate, glutamic acid, pipecolate, gluconic acid, quinate, N-acetyl-glucosamine, indole-3-acetic acid, indole-3-pyruvic acid, maltose, myo-inositol, dehydro-ascorbic acid dimer, 2-aminophenol, 4-aminobenzoate, 3-amino-4-hydroxybenzoic acid, dihydroxymandelic acid, dethiobiotin, 2-methylmaleate, alpha-ketoglutaric acid, 3-methyl-2-oxovaleric acid, hydroquinone, 2-5-dihydroxybenzoate, N-acetylserotonin, itaconate, azelaic acid, pyruvate, diacetyl, phenyl acetate, trans-cinnamaldehyde, adipic acid, suberic acid, propynoate, 2-oxoadipate, benzyl alcohol, n-acetyl-leucine, 2′-4′-dihydroxyacetophenone, 1-methyl-6-7-dihydroxy-1-2-3-4-tetrahydroisoquinoline, 4-quinolinecarboxylic acid, salicylamide, homovanillate, 3-methoxy-4-hydroxymandelate, indole-3-acetaldehyde, 4-methyl-2-oxovaleric acid, 3-hydroxybenzoate, homogentisate, salicylate, 4-methylcatechol, 3-hydroxyphenylacetate, pyridoxal, biotin, pyruvic aldehyde, 3-[4-hydroxyphenyl]lactate, thymidine, 5′-methylthioadenosine, dihydrofolate, glycine, ethanolamine phosphate, glycerate, methionine, orotate, xanthine, adenine, 5-hydroxyindoleacetate, acetyl phosphate, acetoacetate, pyrrole-2-carboxylate, resorcinol monoacetate, ethyl 3-indoleacetate, cortisone, corticosterone, dehydroascorbate, sucrose, arabinose, xylitol, s-carboxymethyl-I-cysteine, gulonic acid, gama-lactone, mannitol, 2-acetamido-2-deoxy-beta-d-glucosylamine, uridine 5′-diphosphate, cellobiose, 2-hydroxy-4-[methylthio]butyric acid, arabitol, mannose, chenodeoxycholate, 4-coumarate, 3alpha-12alpha-dihydroxy-5beta-cholanate, rosmarinic acid, taurolithocholate, 3-3′-5′-triiodothyronine, deoxycholate, tryptophan, lactate, alanine, thymine, fructose 1-6-biphosphate, shikimate, uridine, uridine-5-monophosphate, carnosine, omega-hydroxydodecanoic acid, methyl jasmonate, dimethylbenzimidazole, docosahexaenoic acid, cholate, 1-hydroxy-2-naphthoate, phenylpyruvate, tricosanoic acid, alpha-tocopherol, guanosine, sarcosine, uracil, succinate, aspartate, creatine, hypoxanthine, phenylalanine, 3-4-dihydroxy-phenylalanine, 2′-deoxycytidine 5′-monophosphate, DAMP, nicotinamide mononucleotide, folic acid, tartaric acid, asparagine, homoserine, valine, guanine, lysine, pyridoxine, tyrosine, N-acetyl-tryptophan, guanidinoacetate, 2-phosphoglyceric acid, creatinine, trans-aconitate, isocitric acid, N-acetyl-mannosamine, thiourea, trehalose, alpha-aminoadipate, leucine, glucosamine 6-phosphate, II-2-6-diaminoheptanedioate, theophylline, 3-dehydroshikimate, noradrenaline, and deoxycytidine.

In some embodiments, the method comprises comparing the impact of different RS on microbial (including bacterial) protein and/or RNA expression and/or microbial metabolite production. In some embodiments, the method comprises a metaproteomic analysis. In some embodiments, the metaproteomic analysis is a shotgun metaproteomic analysis. Optionally, metaproteomic profiles are compared using methods known in the art including nonlinear iterative partial least square analysis and pathway enrichment analysis.

In other embodiments, the method comprises the methods described in U.S. Pat. No. 11,175,294 B2 (incorporated by reference).

In some embodiments, the method comprises comparing the impact of different RS on microbial metatranscriptomes.

In some embodiments, the method comprises comparing the impact of different RS on microbial metabolite production.

In some embodiments of the invention, the microbial responses to the individual RS or combinations thereof is predicted using the Rapid Assay of an Individual's Microbiome (called RapidAIM and described in U.S. Pat. No. 11,175,294 B2 incorporated by reference). Briefly, RapidAIM consists of panel of microbiomes derived from one or more individuals that are treated with selected RS and screened in a multi-well format. This approach allows for rapid classification of RS that have no affect or affect the microbiota composition (biota-affectors), or compounds that are affected by the microbiome (biota-altered) using metagenomics, and/or fast-pass metaproteomics, and/or metatranscriptomics, and/or metabolomics assessment.

In some embodiments, the gut microbiota is inoculated and grown in culture media in conditions that replicate lumen environment of a colon or other part of the gut including the small intestine (by using an optimized broth medium which includes inorganic salts, bile salts, mucin, and short-chain fatty acids as described Li et al 2018¹⁹ and Li et al 2020²⁰) that the microbiome sample is harvested from. The method may be performed in any multiwell format (e.g. 6 well to 96 well plate formats, or any other type of format, for example, using tubes or flasks).

To gain a better understanding of the invention described herein, the following examples are set forth. It will be understood that these examples are intended to describe illustrative embodiments of the invention and are not intended to limit the scope of the invention in any way.

### Example 1: Individualized Ex Vivo Resistant Starch Fermentation by Pediatric Inflammatory Bowel Disease Patient Microbiomes

Targeted microbiome modulation via prebiotics is emerging as a common objective in the field of microbiome science¹⁶⁻¹⁸. This goal is more attainable by improvements in high throughput ex vivo microbiome culturing techniques¹⁹, enabling screening an individual's microbiome against multiple types of prebiotics in parallel, such as resistant starches (RS)²⁰.

RS is a category of granular alpha-glucans capable of withstanding hydrolysis by human amylases and not directly absorbed in the gastrointestinal tract. As a component of common foods and supplements, RS possess diverse morphological, physicochemical, and fermentative properties, principally conferred by plant origin and augmentative hydrothermal/chemical processing [reviewed in ¹]. Upon transit to the colon, microbial RS fermentation is considered a collaborative effort initiated by RS-degrading microbes (the primary degraders) liberating glycans to be transformed by cross-feeders, contributing to a cascade of ecosystem remodelling².

Fermentation of dietary fibers, RS included, is studied with an emphasis on short-chain fatty acid (SCFA) production, foremost butyrate³⁻¹⁴. Butyrate plays important roles in maintaining host gut and systemic homeostasis, with potential therapeutic ramifications in conditions such as inflammatory bowel disease (IBD)^{1,15}.

IBD is characterized by complex, dysfunctional host-microbiome interactions²¹⁻²³ serving as an archetypal target for precision microbiome-based therapies. Leveraging RS to activate and restore SCFA production would exemplify the clinical utility of a safe and relatively inexpensive prebiotic therapy^{24,25}. However, realizing such a therapy is hampered by several knowledge gaps.

First, there are highly individualized effects of prebiotics on the microbiome^{26,27}, as well as RS-specific effects at the population level^{3,20}, but these effects are unexplored in the context of IBD.

Second, the focus on butyrate as a primary outcome of prebiotic fermentation marginalizes the broader spectrum of beneficial fermentation metabolites that impact health [reviewed in ²⁸]. Third, clinical studies evaluating fermentation by-products rarely investigate the mechanisms conferring benefit at the individual level.

Hence, the objectives of this study are to investigate RS-specific effects on the microbiome of children with IBD across individuals and within individuals, explore impacts of RS on a broader array of metabolites than solely SCFAs, and conduct systems-level, multi-omic analyses to resolve microbiome functions activated by RS.

### RESULTS

### Resistant Starch Products Vary by Amylolytic Resistance and Morphology

We selected nine commercial RS products to include in our panel of treatments, derived from various plant sources and processing methods. Knowledge of the RS % of each product is essential for proper RS dosing during treatments and depleting the digestible fraction of each product prior to culturing as this fraction would not reach the colon in vivo. However, these products have either unstated RS %, or their RS % varied in the literature or on-label claims, ranging up to 66.9% for a single RS. Therefore, we measured the RS % in each product using a standard method compatible with all nine RS. RS %'s ranged from 46.9% (HiMaize260) to 85.7% (Versafibe1490).

### Resistant Starches are Variably Fermented by Microbiomes From Pediatric Inflammatory Bowel Disease Patients

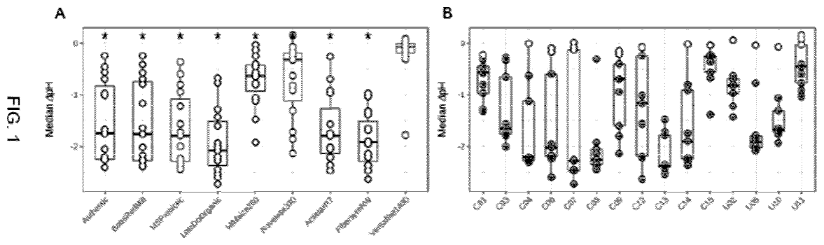

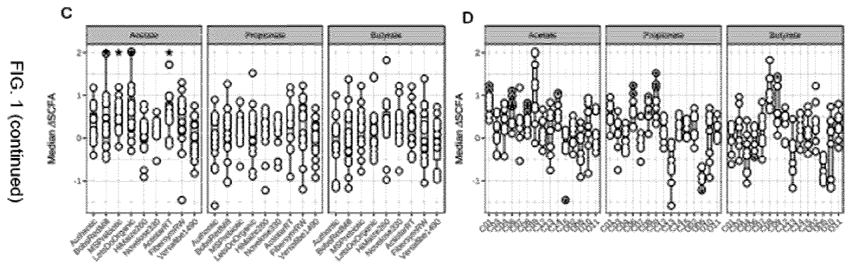

We cultured stool microbiomes from 15 pediatric IBD patients with 9 RS and negative control (PBS) in conditions replicating the human colon lumen¹⁹. After 18 h of culturing, we measured the change in supernatant pH relative to PBS-negative control as a simple proxy for RS fermentation. Significant reductions in pH were observed across all RS except Versafibe 1490 (FIG. 1A), indicating a net acidification of supernatant caused by RS fermentation. Notably, similar RS yielded similar patterns in pH change. At the individual-level, pH responses were highly variable, with all individuals capable of fermenting multiple RS (FIG. 1B).

Short-chain fatty acids are products of the saccharolytic fermentation process and are considered key targets of RS interventions. We measured the concentrations of the three most abundant SCFAs (acetate, propionate, butyrate) in cultured supernatant using an LC-MS approach. Certain RS, including BobsRedMill, MSPrebiotic, LetsDoOrganic, and ActistarRT led to a significantly increased production of acetate, the most abundant SCFA in the human gut (FIG. 1C). Notably, no changes to propionate or butyrate were significant at the group-level for any RS. At the individual level, acetate was increased in 4 individuals (C01, C06, C08, C14) and decreased in 1 (C15 FibersymRW), propionate was increased in 3 (C06, C08, C09) and decreased in 1 (U05 HiMaize260), and butyrate was increased in 1 (C09 LetsDoOrganic) and decreased in 1 (C06 LetsDoOrganic) (FIG. 1D).

After evaluating simple readouts of RS fermentation, we assessed changes to bacterial alpha-diversity in response to RS. Bacterial responses to RS have been well-characterized in the general population [reviewed in¹], but not in pediatric patients with IBD. Wewe performed 16S rRNA gene amplicon sequencing on cultured microbiota and ). found significant increases in microbial richness in response to LetsDoOrganic and potato RS′, and a significant decrease in evenness in response to FibersymRW (FIG. 1E). At the individual-level, significant impacts to alpha-diversity were observed in at least 1 RS in 13/15 individuals (FIG. 1F).

### Resistant Starch Fermentation Remodels Microbiome Compositions

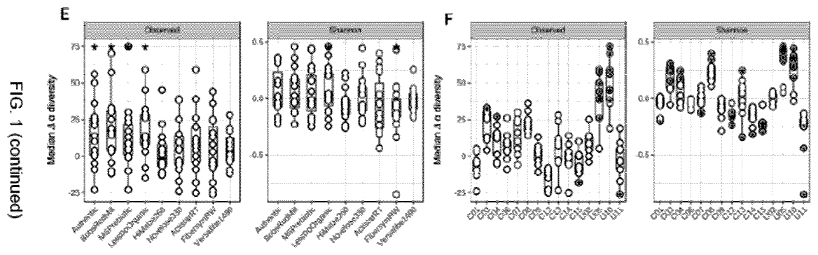

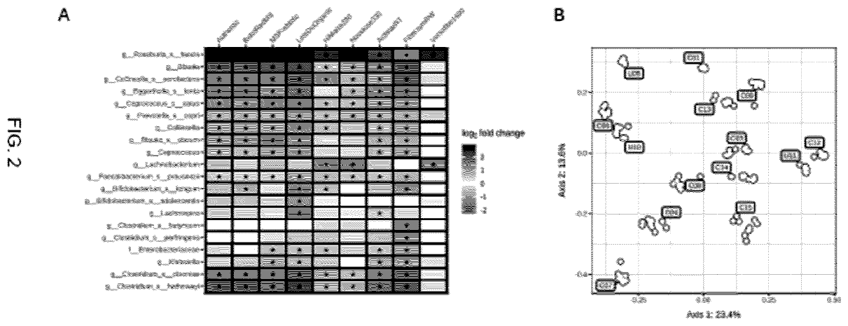

We used MaAsLin2 to examine enrichments and depletions of specific bacteria at the group-level, with a negative control (PBS) serving as the comparison group. After fitting random effects for individual and applying false-discovery rate correction, 69 agglomerated taxa were significantly affected by at least one RS (FIG. 2A).

To assess global changes to microbiome compositions (beta-diversity), we calculated Bray-Curtis dissimilarities, represented via principal coordinate analysis (FIG. 2B). PERMANOVA indicated variance in community structure being primarily driven by individual effects (R²=68.85%, p=0.001) with relatively trivial RS treatment effects (R²=0.61%, p=0.025).

To examine enrichments and depletions of specific bacteria at the individual-level, we repeated the MaAsLin2 analysis for each individual participant. After applying FDR correction, 119 taxa were significantly affected by RS across the 15 individuals). We selected 10 taxa with the lowest and highest Gini impurity of log₂-fold changes, respectively to highlight taxa with the most conserved and discrepant responses across individuals. Consistent with the group-level analysis, Roseburia faecis, Blautia, and Clostridium hathewayi were among taxa with the most conserved responses to RS across individuals (FIG. 2C). Interestingly, two RS primary degraders, B. adolescentis and R. bromii were among the most discrepant taxa (FIG. 2D). Furthermore, mucin-degrading and mucin-dwelling species such as R. gnavus, R. torques, and A. muciniphila were enriched by RS in some individuals and depleted in others, in an RS-dependent manner.

### Resistant Starch Fermentation Remodels Microbiome Functions

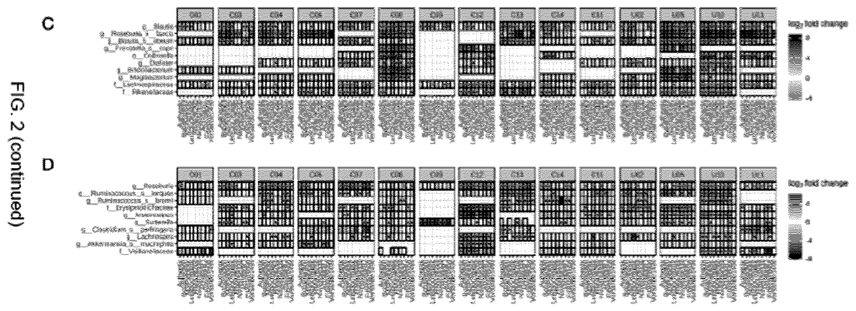

After finding robust changes in microbiome compositions in response to RS, we evaluated microbiome functional changes via shotgun metaproteomics²⁹. We identified 24,794 proteins with 1,638 unique COG annotations. Wewe summed normalized protein intensities by their COG annotation and used MaAsLin2 to perform group-level functional comparisons. We found 90 significantly altered COGs (FIG. 3A). YaaT, a cell fate regulator involved in sporulation, was the only COG enriched by all RS, potentially due to supernatant acidification. Glycosidase/amylase and pullulanase/glycogen debranching enzyme, both involved in starch degradation, were enriched by 6/9 RS and 5/9 RS, respectively. Conversely, depleted COGs included glycerol kinase and glycerol uptake facilitator.

To assess global changes to microbiome functions, we performed principal component analysis on normalized protein intensities. PERMANOVA indicated most variance being explained by individual effects (R²=92.27%, p=0.001) with no significant contribution from RS treatment effects (R²=0.06%, p=0.760) (FIG. 3B).). Individual-wise dispersion was significant (p=0.007), indicating that RS elicits varying effects on individual metaproteomes after scaling feature intensities.

At the individual-level, 1113 unique COGs were identified as significant across 14/15 individuals. Glycosidase/amylase, glycerol kinase, and superoxide dismutase were among COGs impacted most consistently across individuals, however, significant changes were absent in several individuals and RS conditions (FIG. 3C). Among the most discrepant COGs, ABC-type oligopeptide transport system, hemerythrin, and phosphotransferase system components were enriched by RS in some individuals and depleted in others (FIG. 3D).

### Resistant Starch Fermentation Remodels Microbiome Metabolites

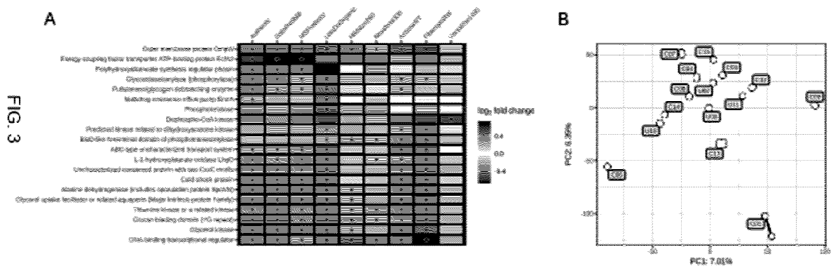

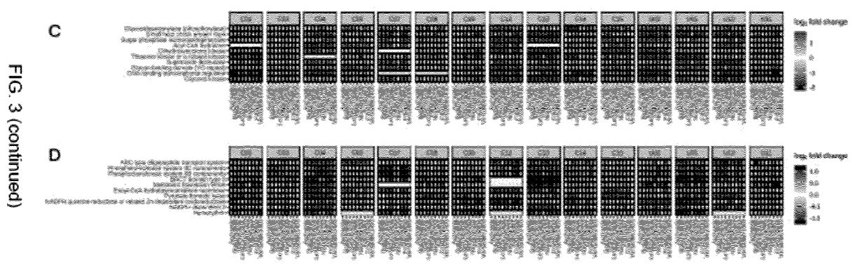

We used semiSemi-targeted metabolomics analysis with a panel of 134 standards to characterize metabolic changes driven by RS fermentation in 13 of the participants. We found 117 metabolites significantly affected by RS (the top 20 metabolites are shown FIG. 4A). Interestingly, 98/117 metabolites were significantly increased by RS, with LetsDoOrganic and FibersymRW driving the greatest number of changes (87 and 80, respectively). 10-hydroxydecanoate (medium chain hydroxy acid) and 3-[2-hydroxyphenyl]-propanoate (phenylpropanoic acid) were enriched by all 9 RS. Uridine, cytidine 2-3-cyclic monophosphate, and N-acetyl-L-cysteine were among the most strongly increased metabolites. Uridine was maximally affected by LetsDoOrganic (log₂-fold change=5.05), and interestingly, strongly correlated with pH change (Spearman rho=−0.81). Furthermore, secondary bile acids deoxycholate and 3alpha-hydroxy-5beta-cholanate (lithocholate), both depleted by RS, were top contributors to metabolome variation after controlling for change in pH.

Global changes to the metabolome were primarily driven by individual variation (R²=82.00%, p=0.001) with >10-fold less variance explained by RS treatment (R²=9.53%, p=0.001) (FIG. 4B).

At the individual-level, all 134 metabolites were significantly impacted across the 13 individuals tested. N-acetyl-L-cysteine, riboflavin, nucleic acid derivatives (adenine, thymidine, deoxyuridine), and quinate are consistently enriched across microbiomes (FIG. 4C). Conversely, nicotinate, fumarate, and L-glutamic acid were among the most discrepantly affected metabolites (FIG. 4D). Notably, nicotinate was identified as significantly enriched by ActistarRT and FibersymRW at the group-level, while potato RS′ drive opposing changes across individuals.

### Integrated Cross-Omic Networks are Resistant Starch- and Individual-Specific

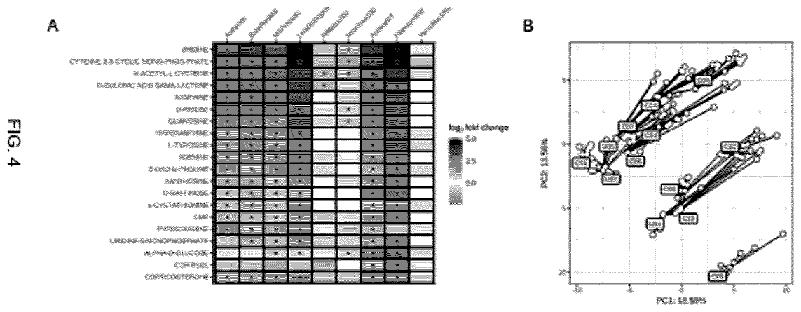

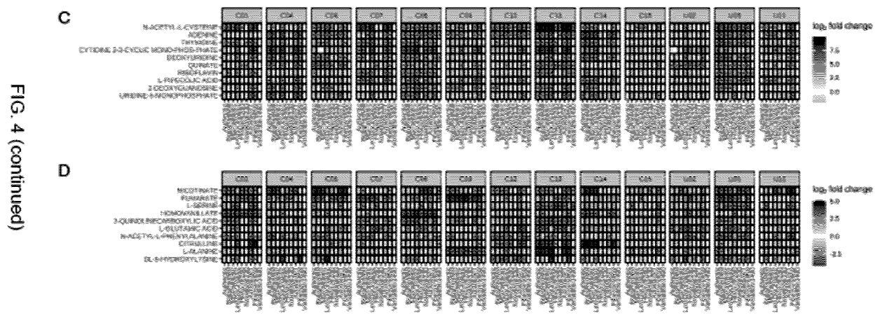

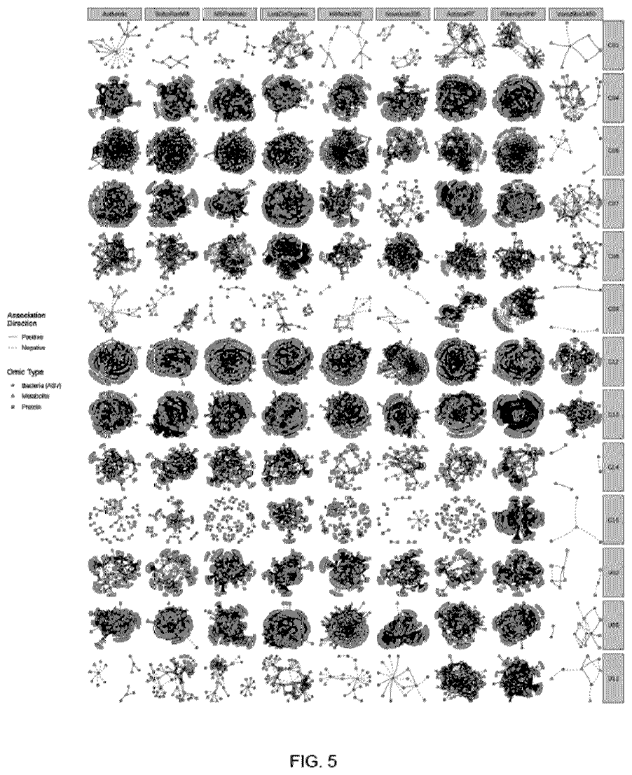

After associating bacteria, their functions, and metabolites with RS fermentation, we sought to resolve pairwise associations between cross-omic features via network analysis. However, the stark individual- and RS-specific discrepancies observed so far would render a confounded network. Hence, we opted to assess associations one RS and individual at a time. Using bacterial ASVs, proteins, and metabolites identified as significantly enriched or depleted (BH-adjusted p<0.05), we present cross-omic associations with significant Spearman correlation coefficients (BH-adjusted p<0.05) in FIG. 5. Each RS and microbiome formed a unique cross-omic network highlighting the need for profiling these interactions in order to identify the ideal RS for each participant.

Bacterial ASVs most embedded in networks (i.e. highest betweenness centrality) include Dorea (C13 LetsDoOrganic), Blautia producta (U05 LetsDoOrganic), and Lachnospiraceae_spp (C13 Authentic). Proteins most embedded in networks, with a COG annotation, include glutamate dehydrogenase (U02 Novelose330), dTDP-glucose pyrophosphorylase (U05 Authentic), and glycerol dehydrogenase (C07 HiMaize260). Metabolites most embedded include N-acetyl-L-phenylalanine (C13 ActistarRT), uracil (U05 LetsDoOrganic), and N-acetyl-L-cysteine (C07 LetsDoOrganic).

### DISCUSSION

We undertook a systems-level examination of ex vivo gut microbiome responses to RS in 15 individuals belonging to a clinically relevant population. Our findings corroborate that individual variation outweighs fiber-specific outcomes^{13,20,27,30,31}.

For example, Roseburia, Blautia, Collinsella, and Eggerthella are genera enriched by RS at the group level. These taxa are often depleted in IBD^{21,35,36}, are associated with better treatment outcomes in treatment-naïve pediatric UC³⁷ and predict treatment success in adult CD^{38,39}. Conversely, we found that R. torques and R. gnavus were enriched by some RS in some individuals and depleted in others. These findings give pause as they were reported to be positively associated with IBD in several meta-analyses^{35,40,41} and suggests the need .for ex vivo screening to ensure the absence of potentially deleterious RS impacts.

Our study illustrates remarkable individual variation in RS fermentation. The numbers of bacteria, proteins, and metabolites significantly affected by RS varied by orders of magnitude across individuals, and among RS within individuals. Our ex vivo system offers a tractable approach to examining these responses. Importantly, our finding of RS-enriched bacterial taxa and metabolites positively and negatively associated with IBD underline the rationale and need for pre-screening RS as a potential therapy in this population.

### Example 2: The Therapeutic Potential of Resistant Starches in Modulating the CD Microbiome

Crohn's disease (CD) is a debilitating chronic disease without a cure and with limited treatment options. The current treatment paradigm aims to bring active disease into remission and then maintain remission using various combinations of a broad number of anti-inflammatory and immunomodulatory drugs due to variability in patient responses. Therapies use a systemic “brute force” approach to alleviate inflammation and thereby symptoms and complications, but do not target the underlying causes of disease. Current therapeutics can be effective but can also have significant side-effects, there are no prospects for stopping medication and patients still experience variable outcomes, morbidities, and complications. Thus, there is a need for alternative CD therapies. A role for the gut microbiome in stimulating inflammation in CD patients though a potentially “leaky-gut” has been suggested. However, none of the current therapies in use are designed to modulate the gut microbiome. As such, therapies, like resistant starches (RS) supplementation, that could restore a healthy intestinal microbiome would be transformative as they could reduce gut permeability with subsequent reduction of the stimuli that activate inflammation thereby reducing systemic inflammation.

We first carried out a systematic review and meta-analysis to summarize the preclinical and clinical effects of RS, with the primary objective to assess and demonstrate clinical safety in IBD. The systematic review demonstrated that RS is associated with reduced histological damage in animal studies, and improvements in IBD patient clinical remission²⁵. However, these results need to be tempered by potential study biases and by statistical and methodological heterogeneity. Notably, human trials showed significant variability in treatment effect. These varied outcomes are likely due to each RS having a different interaction with each individual's microbiota composition and/or functionality. A personalized dietary intervention that matches specific RS to each individual microbiota might be required for consistent beneficial effects. An intervention protocol to conduct personalized RS treatment in CD patients was established. A panel of 9 diverse RS was assembled, and an intervention trial protocol was developed and initiated (ClinicalTrials.gov: NCT04522271).

The strategy to identify the optimal RS to prescribe as a therapeutic adjuvant entails:
1. Determining the effect of the various RS on a patient's microbiome using RapidAIM;
2. Comparing RS treated patient microbiomes to those obtained from non-IBD control patients; and
3. Selecting the optimal RS based on its efficacy to boost butyrate producing microbes and shift patient microbiomes towards non-IBD controls.

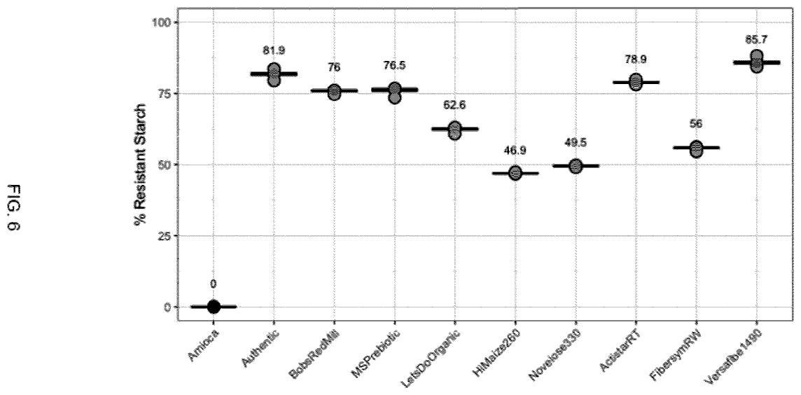

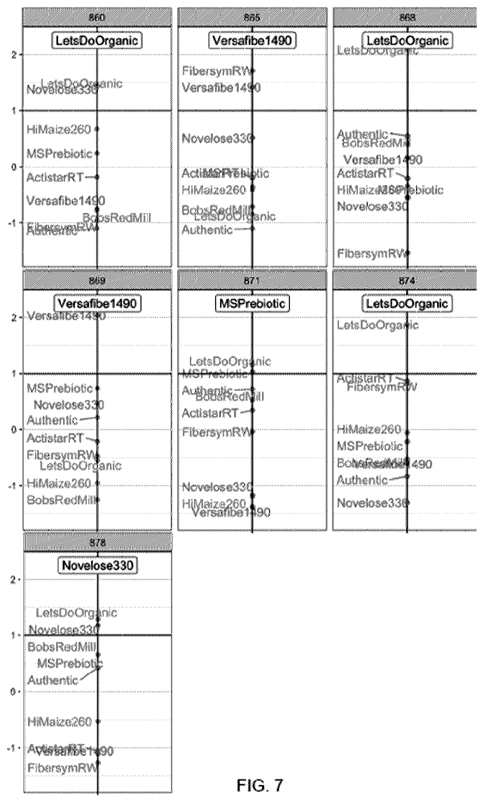

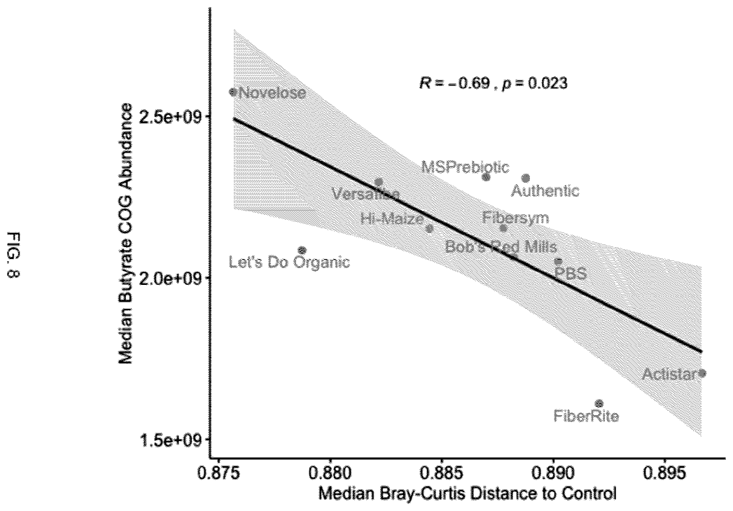

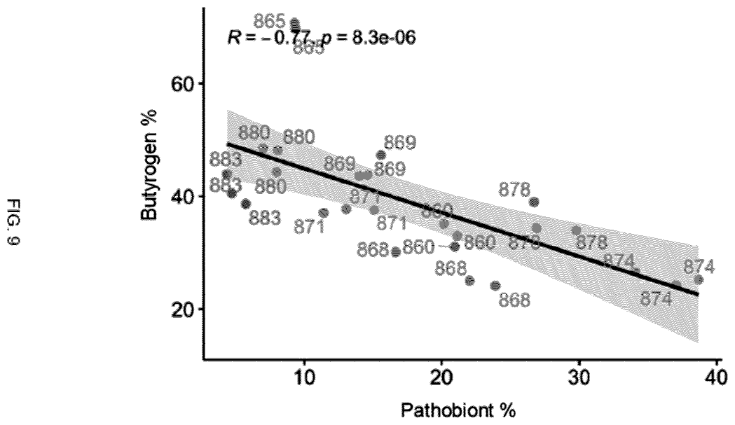

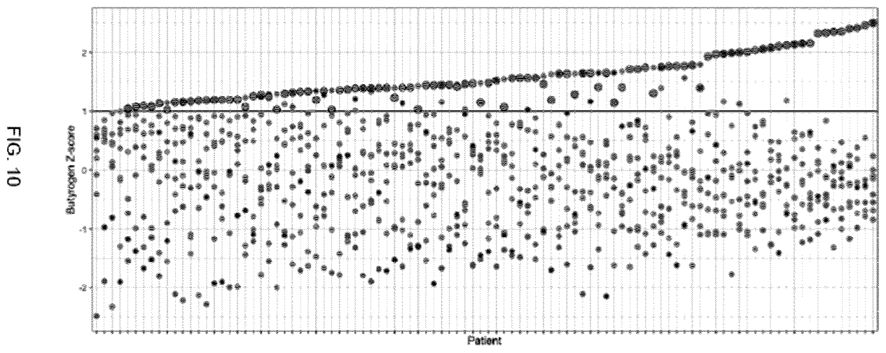

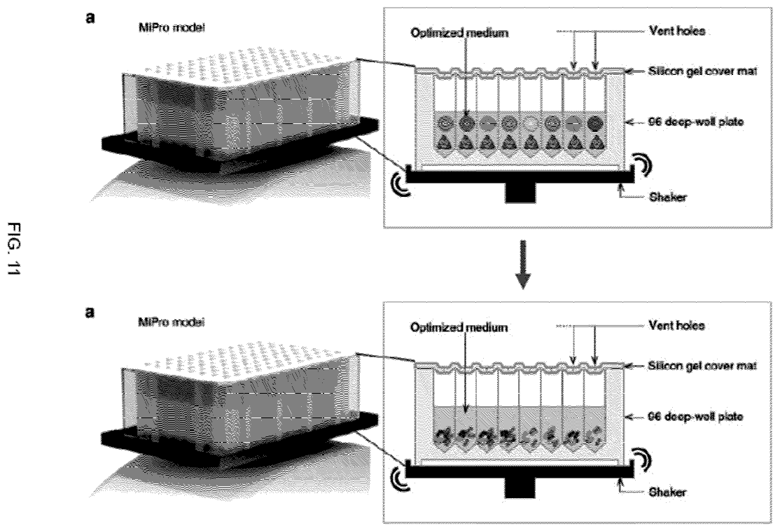

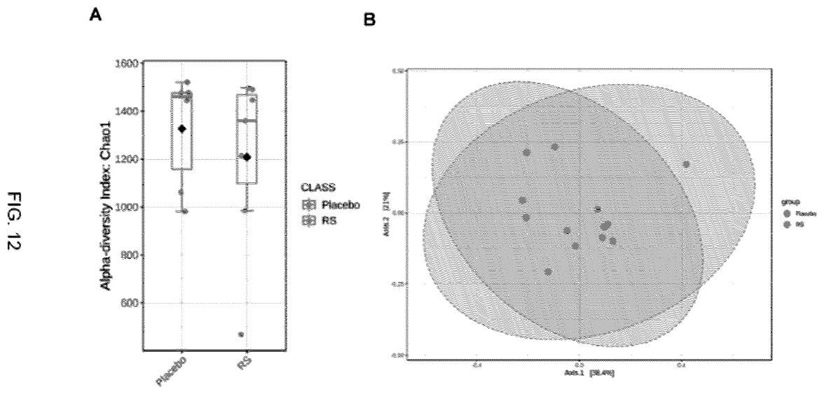

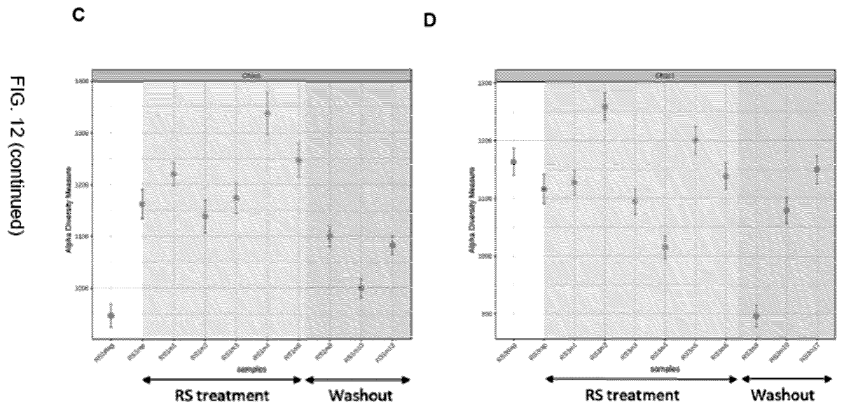

The results show that the microbiota responds differently to each RS. FIG. 7 represents the results from the RapidAIM assay in selecting the optimal RS for nine CD patients. As expected and supporting the hypothesis, the optimal RS varies between patients confirming that RS ought to be individualized. Interestingly, the expression of butyrate biogenic enzymes correlated negatively with the Bray-Curtis dissimilarity between control and patients' microbiome (FIG. 8) in agreement with the decrease of butyrate-producing microbes in IBD patients. Furthermore, across all samples, there is a strong and significant negative Spearman correlation (FIG. 9) between butyrate-producers and pathobionts that are known to be associated with CD disease severity. This observation indicates that the increase in butyrate producers through personalized RS therapy has the potential to outcompete known pathobionts and thus to restore microbiome homeostasis.

We evaluated the efficacy of personalized RS treatment on new-onset CD patients (ClinicalTrials.gov: NCT04522271) by conducting a double-blinded placebo controlled intervention trial. New onset CD patients with terminal ileal +/− colonic disease were identified at diagnostic endoscopy. After patients achieved initial remission following induction therapy with corticosteroids (CS; typically, 3 to 4 weeks post-diagnosis), each participant's stool microbiota was collected and processed through our RS-RapidAIM pipeline to determine the optimal RS for generating a healthier microbiota (as described above). The optimal RS was introduced as an adjuvant following this induction of remission with corticosteroids (CS). CS tapering began thereafter to discontinuation along with the addition of a concomitant immunomodulator (IMM) maintenance therapy according to standard clinical practice. The optimal RS (defined as the RS that increases the relative abundance of butyrate-producers and minimizes the distance of participant microbiomes to non-IBD controls) was introduced as an adjuvant for up to 6 months. Containers with daily doses of the RS formulations were provided to the participant. We utilized a double-blinded approach with both placebo/RS formulations tasting and looking the same. Previous human intervention trials have shown that RS supplementation is safe with dose-related elevated incidence of flatulence and “gut rumbling” as the main reported adverse events. The dose was determined based on the patient's body surface area [BSA] and we prescribed 7.5 g RS/m² daily (rounded to the nearest gram) for up to 6 months follow by a washout period of 6 months.

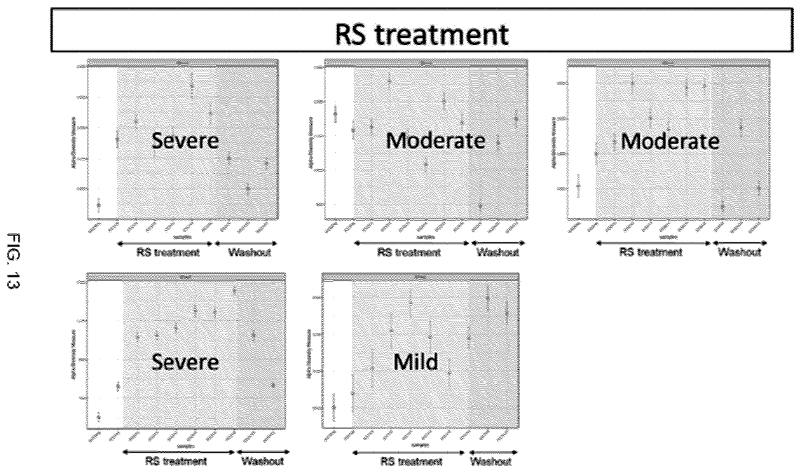

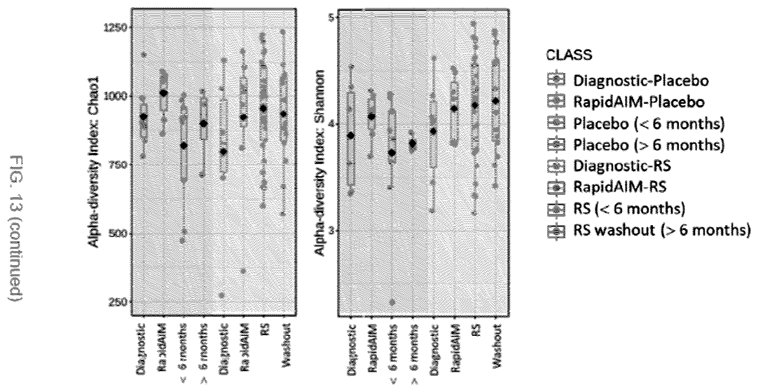

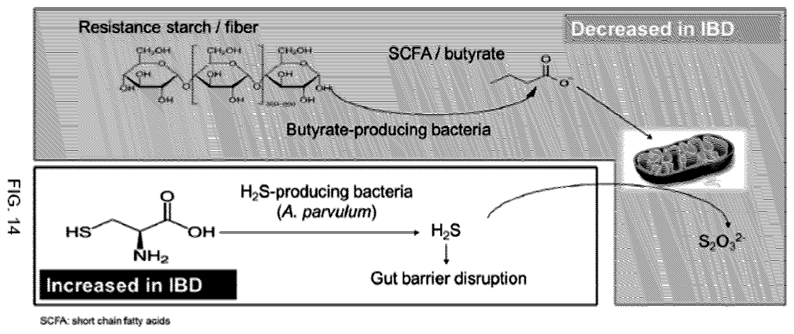

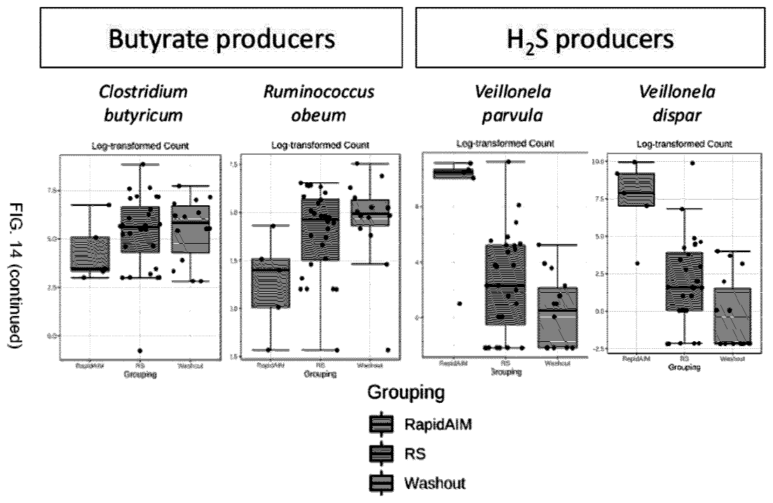

The goal of these this trial (single center, randomized, placebo-controlled, double-blinded, parallel, pilot clinical trials) was to assess if a personalized RS will target the underlying cause of IBD and restore a “healthier” gut microbiome in pediatric CD participants. The personalized RS intervention significantly increased bacterial diversity (FIG. 12-13), the relative abundance of butyrate producers and beneficial microbes (e.g., Faecalibacterium, Ruminococcus, Phascolarctobacterium, Collinsella, and Blautia) while decreasing the relative abundance of pathobionts (e.g. Veillonella, Haemophilus and Streptococcus) (FIG. 14). In comparison, the microbiome of placebo patients showed a significant decrease in these butyrate producers over time with a corresponding increase in these aforementioned pathobionts. These results indicate that the hypothesis was correct and the personalized RS intervention prescribed from the RapidAIM ex vivo assay coupled with the bioinformatic analysis had indeed shifted the IBD microbiome toward a “healthier” microbiome.

### REFERENCES

1. Dobranowski PA, Stintzi A. Resistant starch, microbiome, and precision modulation. Gut Microbes. 2021;13(1):1926842. doi:10.1080/19490976.2021.1926842
2. Cerqueira FM, Photenhauer AL, Pollet RM, Brown HA, Koropatkin NM. Starch Digestion by Gut Bacteria: Crowdsourcing for Carbs. Trends Microbiol. 2020;28(2):95-108. doi:10.1016/j.tim.2019.09.004
3. Deehan EC, Yang C, Perez-Muñoz ME, et al. Precision Microbiome Modulation with Discrete Dietary Fiber Structures Directs Short-Chain Fatty Acid Production. Cell Host Microbe. 2020;27(3):389-404.e6. doi:10.1016/j.chom.2020.01.006
4. Venkataraman A, Sieber JR, Schmidt AW, Waldron C, Theis KR, Schmidt TM. Variable responses of human microbiomes to dietary supplementation with resistant starch. Microbiome. 2016;4(1):33. doi:10.1186/s40168-016-0178-x
5. Wang S, Zhang B, Chen T, Li C, Fu X, Huang Q. Chemical Cross-Linking Controls in Vitro Fecal Fermentation Rate of High-Amylose Maize Starches and Regulates Gut Microbiota Composition. J Agric Food Chem. 2019;67(49):13728-13736. doi:10.1021/acs.jafc.9b04410
6. Belenguer A, Duncan SH, Calder AG, et al. Two routes of metabolic cross-feeding between Bifidobacterium adolescentis and butyrate-producing anaerobes from the human gut. Appl Environ Microbiol. 2006;72(5):3593-3599. doi:10.1128/AEM.72.5.3593-3599.2006
7. Poole AC, Goodrich JK, Youngblut ND, et al. Human Salivary Amylase Gene Copy Number Impacts Oral and Gut Microbiomes. Cell Host Microbe. 2019;25(4):553-564.e7. doi:10.1016/j.chom.2019.03.001
8. Giuberti G, Gallo A. In vitro evaluation of fermentation characteristics of type 3 resistant starch. Heliyon. 2020;6(1):e03145. doi:10.1016/j.heliyon.2019.e03145
9. Abell GCJ, Cooke CM, Bennett CN, Conlon MA, McOrist AL. Phylotypes related to Ruminococcus bromii are abundant in the large bowel of humans and increase in response to a diet high in resistant starch. FEMS Microbiol Ecol. 2008;66(3):505-515. doi:10.1111/j.1574-6941.2008.00527.x
10. Alfa MJ, Strang D, Tappia PS, et al. A randomized trial to determine the impact of a digestion resistant starch composition on the gut microbiome in older and mid-age adults. Clin Nutr Edinb Scotl. 2018;37(3):797-807. doi:10.1016/j.clnu.2017.03.025
11. So D, Yao CK, Gill PA, Pillai N, Gibson PR, Muir JG. Screening dietary fibres for fermentation characteristics and metabolic profiles using a rapid in vitro approach: implications for irritable bowel syndrome. Br J Nutr. 2021;126(2):208-218. doi:10.1017/S0007114520003943
12. McOrist AL, Miller RB, Bird AR, et al. Fecal butyrate levels vary widely among individuals but are usually increased by a diet high in resistant starch. J Nutr. 2011;141(5):883-889. doi:10.3945/jn.110.128504
13. Teichmann J, Cockburn DW. In vitro Fermentation Reveals Changes in Butyrate Production Dependent on Resistant Starch Source and Microbiome Composition. Front Microbiol. 2021;12:640253. doi:10.3389/fmicb.2021.640253
14. Cummings JH, Beatty ER, Kingman SM, Bingham SA, Englyst HN. Digestion and physiological properties of resistant starch in the human large bowel. Br J Nutr. 1996;75(5):733-747. doi:10.1079/bjn19960177
15. Hodgkinson K, El Abbar F, Dobranowski P, et al. Butyrate's role in human health and the current progress towards its clinical application to treat gastrointestinal disease. Clin Nutr. Published online November 2, 2022. doi:10.1016/j.clnu.2022.10.024
16. Gibbons SM, Gurry T, Lampe JW, et al. Perspective: Leveraging the Gut Microbiota to Predict Personalized Responses to Dietary, Prebiotic, and Probiotic Interventions. Adv Nutr. 2022;13(5):1450-1461. doi:10.1093/advances/nmac075
17. DeMartino P, Cockburn DW. Resistant starch: impact on the gut microbiome and health. Curr Opin Biotechnol. 2020;61:66-71. doi:10.1016/j.copbio.2019.10.008
18. Bendiks ZA, Knudsen KEB, Keenan MJ, Marco ML. Conserved and variable responses of the gut microbiome to resistant starch type 2. Nutr Res N Y N. 2020;77:12-28. doi:10.1016/j.nutres.2020.02.009
19. Li L, Ning Z, Zhang X, et al. RapidAIM: a culture- and metaproteomics-based Rapid Assay of Individual Microbiome responses to drugs. Microbiome. 2020;8(1):33. doi:10.1186/s40168-020-00806-z
20. Li L, Ryan J, Ning Z, et al. A functional ecological network based on metaproteomics responses of individual gut microbiomes to resistant starches. Comput Struct Biotechnol J. 2020;18:3833-3842. doi:10.1016/j.csbj.2020.10.042
21. Mottawea W, Chiang CK, Mühlbauer M, et al. Altered intestinal microbiota-host mitochondria crosstalk in new onset Crohn's disease. Nat Commun. 2016;7:13419. doi:10.1038/ncomms13419
22. Cleynen I, Boucher G, Jostins L, et al. Inherited determinants of Crohn's disease and ulcerative colitis phenotypes: a genetic association study. Lancet Lond Engl. 2016;387(10014):156-167. doi:10.1016/S0140-6736(15)00465-1
23. Lloyd-Price J, Arze C, Ananthakrishnan AN, et al. Multi-omics of the gut microbial ecosystem in inflammatory bowel diseases. Nature. 2019;569(7758):655-662. doi:10.1038/s41586-019-1237-9
24. Healey GR, Celiberto LS, Lee SM, Jacobson K. Fiber and Prebiotic Interventions in Pediatric Inflammatory Bowel Disease: What Role Does the Gut Microbiome Play? Nutrients. 2020;12(10):E3204. doi:10.3390/nu12103204
25. Montroy J, Berjawi R, Lalu MM, et al. The effects of resistant starches on inflammatory bowel disease in preclinical and clinical settings: a systematic review and meta-analysis. BMC Gastroenterol. 2020;20(1):372. doi:10.1186/s12876-020-01516-4
26. Holmes ZC, Villa MM, Durand HK, et al. Microbiota responses to different prebiotics are conserved within individuals and associated with habitual fiber intake. Microbiome. 2022;10(1):114. doi:10.1186/s40168-022-01307-x
27. Cantu-Jungles TM, Bulut N, Chambry E, et al. Dietary Fiber Hierarchical Specificity: the Missing Link for Predictable and Strong Shifts in Gut Bacterial Communities. mBio. 2021;12(3):e0102821. doi:10.1128/mBio.01028-21
28. Lavelle A, Sokol H. Gut microbiota-derived metabolites as key actors in inflammatory bowel disease. Nat Rev Gastroenterol Hepatol. 2020;17(4):223-237. doi:10.1038/s41575-019-0258-z
29. Cheng K, Ning Z, Zhang X, et al. MetaLab: an automated pipeline for metaproteomic data analysis. Microbiome. 2017;5(1):157. doi:10.1186/s40168-017-0375-2
30. Lancaster SM, Lee-McMullen B, Abbott CW, et al. Global, distinctive, and personal changes in molecular and microbial profiles by specific fibers in humans. Cell Host Microbe. 2022;30(6):848-862.e7. doi:10.1016/j.chom.2022.03.036
31. Bonazzi E, Bretin A, Vigué L, et al. Individualized microbiotas dictate the impact of dietary fiber on colitis sensitivity. Microbiome. 2024;12(1):5. doi:10.1186/s40168-023-01724-6
32. Romero Marcia AD, Yao T, Chen MH, Oles RE, Lindemann SR. Fine Carbohydrate Structure of Dietary Resistant Glucans Governs the Structure and Function of Human Gut Microbiota. Nutrients. 2021;13(9):2924. doi:10.3390/nu13092924
33. Hamaker BR, Tuncil YE. A perspective on the complexity of dietary fiber structures and their potential effect on the gut microbiota. J Mol Biol. 2014;426(23):3838-3850. doi:10.1016/j.jmb.2014.07.028
34. Debray R, Herbert RA, Jaffe AL, Crits-Christoph A, Power ME, Koskella B. Priority effects in microbiome assembly. Nat Rev Microbiol. 2022;20(2):109-121. doi:10.1038/s41579-021-00604-w
35. Su Q, Liu Q, Lau RI, et al. Faecal microbiome-based machine learning for multi-class disease diagnosis. Nat Commun. 2022;13(1):6818. doi:10.1038/s41467-022-34405-3
36. Galipeau HJ, Caminero A, Turpin W, et al. Novel Fecal Biomarkers That Precede Clinical Diagnosis of Ulcerative Colitis. Gastroenterology. 2021;160(5):1532-1545. doi:10.1053/j.gastro.2020.12.004
37. van de Guchte M, Mondot S, Doré J. Dynamic Properties of the Intestinal Ecosystem Call for Combination Therapies, Targeting Inflammation and Microbiota, in Ulcerative Colitis. Gastroenterology. 2021;161(6):1969-1981.e12. doi:10.1053/j.gastro.2021.08.057
38. Yilmaz B, Juillerat P, Øyås O, et al. Microbial network disturbances in relapsing refractory Crohn's disease. Nat Med. 2019;25(2):323-336. doi:10.1038/s41591-018-0308-z
39. Lee JWJ, Plichta D, Hogstrom L, et al. Multi-omics reveal microbial determinants impacting responses to biologic therapies in inflammatory bowel disease. Cell Host Microbe. 2021;29(8):1294-1304.e4. doi:10.1016/j.chom.2021.06.019
40. Ning L, Zhou YL, Sun H, et al. Microbiome and metabolome features in inflammatory bowel disease via multi-omics integration analyses across cohorts. Nat Commun. 2023;14(1):7135. doi:10.1038/s41467-023-42788-0
41. Vestergaard MV, Allin KH, Eriksen C, et al. Gut microbiota signatures in inflammatory bowel disease. United Eur Gastroenterol J. 2024;12(1):22-33. doi:10.1002/ueg2.12485
42. Chen R, Zhang C, Xu F, et al. Meta-analysis reveals gut microbiome and functional pathway alterations in response to resistant starch. Food Funct. 2023;14(11):5251-5263. doi:10.1039/d3fo00845b
43. Turpin W, Lee SH, Raygoza Garay JA, et al. Increased Intestinal Permeability Is Associated With Later Development of Crohn's Disease. Gastroenterology. 2020;159(6):2092-2100.e5. doi:10.1053/j.gastro.2020.08.005
44. Leibovitzh H, Lee SH, Xue M, et al. Altered Gut Microbiome Composition and Function Are Associated With Gut Barrier Dysfunction in Healthy Relatives of Patients With Crohn's Disease. Gastroenterology. 2022;163(5):1364-1376.e10. doi:10.1053/j.gastro.2022.07.004
45. Miquel S, Leclerc M, Martin R, et al. Identification of metabolic signatures linked to anti-inflammatory effects of Faecalibacterium prausnitzii. mBio. 2015;6(2):e00300-15. doi:10.1128/mBio.00300-15
46. Liu PS, Wang H, Li X, et al. α-ketoglutarate orchestrates macrophage activation through metabolic and epigenetic reprogramming. Nat Immunol. 2017;18(9):985-994. doi:10.1038/ni.3796
47. Tian Q, Bravo Iniguez A, Sun Q, Wang H, Du M, Zhu MJ. Dietary Alpha-Ketoglutarate Promotes Epithelial Metabolic Transition and Protects against DSS-Induced Colitis. Mol Nutr Food Res. 2021;65(7):2000936. doi:10.1002/mnfr.202000936
48. Noecker C, Sanchez J, Bisanz JE, et al. Systems biology elucidates the distinctive metabolic niche filled by the human gut microbe Eggerthella lenta. PLoS Biol. 2023;21(5):e3002125. doi:10.1371/journal.pbio.3002125
49. Jeengar MK, Thummuri D, Magnusson M, Naidu VGM, Uppugunduri S. Uridine Ameliorates Dextran Sulfate Sodium (DSS)-Induced Colitis in Mice. Sci Rep. 2017;7(1):3924. doi:10.1038/s41598-017-04041-9
50. Kim EK, Lee KA, Hyeon DY, et al. Bacterial Nucleoside Catabolism Controls Quorum Sensing and Commensal-to-Pathogen Transition in the Drosophila Gut. Cell Host Microbe. 2020;27(3):345-357.e6. doi:10.1016/j.chom.2020.01.025
51. Grasberger H, Magis AT, Sheng E, et al. DUOX2 variants associate with preclinical disturbances in microbiota-immune homeostasis and increased inflammatory bowel disease risk. J Clin Invest. 2021;131(9):e141676, 141676. doi:10.1172/JCI141676
52. Marcelino VR, Welsh C, Diener C, et al. Disease-specific loss of microbial cross-feeding interactions in the human gut. Nat Commun. 2023;14(1):6546. doi:10.1038/s41467-023-42112-w

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

We claim:

1. A method of selecting prebiotic nutrition therapy for a subject having inflammatory bowel disease, the method comprising:
a. providing a plurality of gut microbiome samples, optionally stool samples, from one or more subjects;
b. culturing the plurality of microbiome samples in conditions that replicate the lumen environment of a colon or small intestine;
c. supplement each microbiome sample in the plurality of microbiome samples with a pre-digested prebiotic for a period of time;
d. analyze change microbiome in response to the pre-digested prebiotic; and
e. select the optimal prebiotic nutrition based on the analyzed changes.

2. The method of claim 1, wherein step d comprises metabolomic, metaproteomic, 16S rRNA gene, metatranscriptomic and/or metagenomic analysis.

3. The method of claim 2, wherein the metabolomic analysis is semi-targeted.

4. The method of claim 3, wherein the semi-targeted metabolomic analysis includes analysis for acetate, propionate and butyrate.

5. The method of any one of claims 1 to 4, wherein metagenomic analysis comprises 16S rRNA gene analysis.

6. The method of any one of claims 1 to 5, wherein the metaproteomic analysis is a shotgun metaproteomics analysis.

7. The method of any one of claims 1 to 6, wherein the prebiotic nutrition is selected to increase butyrate producing microbes.

8. The method of any one of claims 1 to 7, wherein the prebiotic nutrition is selected to increase the abundance of beneficial microbes.

9. The method of claim 8, wherein the beneficial microbes are selected from Lachnospiraceae, Blautia, Roseburia, Eubacterium, Ruminococcus, Clostridium, and Faecalibacterium; and combinations thereof.

10. The method of any one of claims 1 to 9, wherein the prebiotic nutrition is selected to decrease the abundance of pathobionts.

11. The method of claim 10, wherein the pathobionts are selected from Veillonella, Atopobium, Fusobacterium, Leptotrichia, Prevotella, and Streptococcus; and combinations thereof.

12. The method of any one of claims 1 to 11, wherein steps (a) to (e) are reiterated.

13. The method of claim 12, wherein steps (a) to (e) are reiterated at fixed intervals.

14. The method of claim 12, wherein steps (a) to (e) are reiterated when there is a change in participant's health.

15. The method of any one of claims 1 to 14, wherein the subject is a pediatric subject.

16. The method of any one of claims 1 to 14, wherein the subject is an adult subject.

17. The method of any one of claims 1 to 16, wherein the inflammatory bowel disease is Crohn's Disease.

18. The method of any one of claims 1 to 16, wherein the inflammatory bowel disease is Ulcerative colitis.

19. The method of any one of claims 1 to 18, wherein the prebiotic nutrition therapy is dietary supplementation with resistant starch.

20. A method of treating inflammatory bowel disease in a subject comprising administering a therapeutically effective amount of resistant starch, wherein the resistant starch is selected by a method comprising:
a. culturing a plurality of microbiome samples from the subject in conditions that replicate lumen environment of a colon or small intestine;
b. supplementing each microbiome sample in the plurality of microbiome samples with a prebiotic for a period of time;
c. analyzing change microbiome in response to the prebiotic; and
d. select prebiotic nutrition based on the analyzed change.
